(19) United States
(12) Plant Patent Application Publication
(10) Pub. No.: US 2003/0074707 P1
(43) Pub. Date: Apr. 17, 2003

(54) ''Eleanor Byrd''

(76) Inventor: Charles F. Heidgen, Batavia, IL (US)

Correspondence Address: Charles F. Heidgen, 821 Walnut Street, Batavia, IL, 60510, US

(21) Appl. No.: 09/969,352

(22) Filed: Oct. 3, 2001

(51) Int. Cl. A01H 5/00

(52) U.S. Cl. PLT/325

(57) ABSTRACT

A seedling geranium plant named ‘Eleanor Byrd’, from a self of Light Pink Bird's Egg characterized by its bushy growth habit; a prolific blooming characteristic; and its medium-sized, medium green foliage with definite zonation. The flowers are single, having five petals, and uniquely large with a medium pink color that becomes paler toward the center and an extremely distinct speckling of a deep pink color on each of the petals.

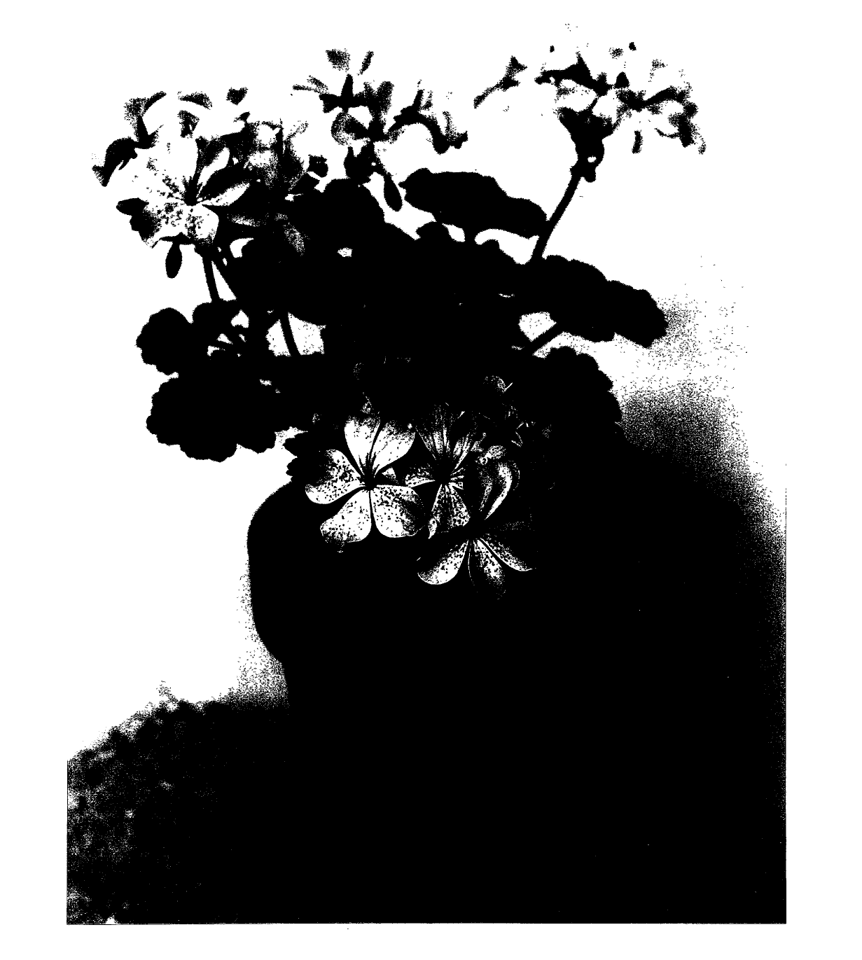

### BACKGROUND OF THE INVENTION

The present invention, hereinafter referred to by the cultivar name “Eleanor Byrd” relates to a new and distinct geranium. “Eleanor Byrd” is a new and improved plant resulting from a planned breeding program as performed by me in my greenhouse at 821 Walnut Street, Batavia, Ill. My breeding program began in March 1977, and has been ongoing since that time. This seedling was originally identified in my records by 87 S 106 and is a seedling that is derived from a self of Light Pink Bird's Egg, an unpatented, novelty flowered, zonal geranium. The botanical classification Pelargonium×hortorum would be appropriate for this plant.

Upon recognition of the outstanding characteristics of this plant, it was selected from the progeny of the stated cross, and was set aside for further observation and testing and was subsequently named “Eleanor Byrd.” Asexual reproduction of “Eleanor Byrd” has been repeatedly accomplished by the rooting of vegetative cuttings in my greenhouse in Batavia, Ill. Examination of the plants so propagated through a series of propagations has demonstrated that the combination of characteristics herein disclosed for “Eleanor Byrd” are firmly fixed and retained through successive generations through asexual reproduction.

### SUMMARY OF THE INVENTION

The new and distinguishing characteristics of “Eleanor Byrd” reside primarily in its bushy growth habit, its prolific blooming characteristic and its medium-sized, medium green foliage with definite zonation. This geranium also has medium to large single flowers with a unique medium pink color that becomes lighter at the center and has a very distinctive speckling of deep pink on each petal. This plant might be seen as a novel and desirable introduction within the industry by a combination of desired traits including:

Being particularly adapted to culture in the ground or in pots due to its bushy growth habit, and its tendency to branch prolifically. Forming a bushy plant mass in approximately 7-9 weeks from one plantlet, and a plant that is not stretched or leggy, but instead, full and bushy.

Having a generous amount of foliage, with medium green coloration and conspicuous zonation over the medium sized leaves and of average density.

Presenting an unusually high number of well filled umbels of varied states of maturity placed well above the foliage mass, and having a medium pink floret coloration, with a slightly lighter pink center and extremely distinctive deep pink speckling pattern, much like a bird's egg, contrastingly highlighted over and against the medium green foliage.

Having a large number of umbels with a generous number of open, single petal florets of a medium to large size and appearance, borne on pedicels which present the florets in a loose arrangement to form umbels having a somewhat open, globular shape.

Presenting a large number of umbels which also vary in stage of maturity, which gives the plant an attractive appearance for an extended period of time, rather than being one that blooms in discontinuous flushes.

The color definitions to follow have been taken from The Royal Horticultural Colour Chart of The Royal Horticultural Society, London, where presented by the letters RHS followed by a color grouping indication, the number indicating the color patch and the letter indicating the shade, except where color definitions in general terms of ordinary meaning are presented. Plant descriptions were made from specimens grown under greenhouse conditions during daylight hours in Batavia, Ill.

### BRIEF DESCRIPTION OF THE DRAWING

The photograph depicts a market unit of a market stage specimen of the plant growing in a 5 ½″ pot, in a stage of inflorescence. The assemblage is shown in a side view, depicting the characteristics of branching, leaves, leaf coloration and zonation, the number and varied stages of buds and flowers, and the positioning of the flower heads relative to the mass of the plants in the pot.

### BOTANICAL DESCRIPTION OF THE PLANT

Botanical classification: Pelargonium×hortorum

Market class: Zonal Geranium suitable for planting in the ground or in containers.

Market name: “Eleanor Byrd”.

Growth: “Eleanor Byrd” is a prolific blooming Geranium with single flowers and the truss or flower head is of medium to large size. The growth is upright in habit and given to branching freely. Growth rate: Average, producing a typical 4 1/2″ flowering geranium in about 7 to 9 weeks from one 2 1/4″ plantlet using standard commercial culture.

Blooming habit: Exceptionally profuse and continuously blooming plant throughout the year, outdoors during the frost-free period, or in the greenhouse; a mature 4 1/2″ pot commonly producing a range of 3 to 6 flower heads at one time, in varying states of maturity.

### INFLORESCENCE

Flower heads

Size.—3 1/4to 3 3/4 inches across when the florets are fully open.

Count.—3 to 6 present at one time.

Truss:

Size.—Medium to large size; ranges from 3 1/8 to 3 15/16 inches in diameter.

Shape.—Rather loose, a flattened globe as it opens.

Number of florets.—Variable, range from approxiamtely 10 to 28, average 14 to 22.

Lasting quality.—Average. Blooms last 4 to 8 days, possibly longer in cooler, dry weather.

Buds:

Sepals.—Shape- Long, average width, pointed. Color- RHS Green Group, 144 C.

Color.—RHS Green Group, 143 B.

Form.—Above average length, average thickness, slight point.

Florets:

Number.—14 to 22.

Form.—Slightly cupped to flat, as it opens fully.

Size.—Ranging from 7/8 inch to 1 3/4 inches across a fully open floret.

Petals:

Number.—Five.

Size.—Ranging from about 3/8 to 7/8 inches long and approxiamtely 1/4 to 5/8 inches across.

Texture.—Soft.

Appearance.—Bright.

Color.—RHS Red Purple Group, 62 C main part of the petal, fading to RHS Red Purple Group 65 D, with tiny, pin point sized speckles of RHS Red Purple Group 57 D, throughout.

Petaloids:

Number.—Substantially absent, but up to 5 may occur.

Shape.—Variable, often misshapen.

Color.—Very similar to that of the petals.

Size.—1/8 to 5/16 inches long and 3/8 to 3/4 inches long.

Sepals

Number.—Usually 5.

Color.—RHS Yellow Green Group, 144 C with a faint veining of RHS Greyed Red Group 178 B.

Pedicel

Length.—About 3/4 to 1 1/8 inches long, with a small knuckle-like nodule located approximately 1/3 of the distance from the joint of the pedicel to the joint of the floret.

Color.—RHS Greyed Red Group, 178 A.

Peduncle

Arises.—From node, opposed to leaf petiole.

Pubescence.—Normal.

Color.—RHS Yellow Green Group, 144 A.

### Reproductive organs

Androecium

Stamens.—Number- 5 to 7. Shape—Ribbon-like filaments jointed at bases. Color—Rose pink. Anther Attachment—versatile.

Anthers.—Number—5 to 7. Shape—Normal, well developed. Color—Brown.

Gynoecium

Pistil.—Number—One, compound. Length—Within the range of 1/16 to 1/8 inches.

Stigma.—Color—Pink.

Style.—Color—Deep pink.

Ovary.—Size—Approximately 1/32 to 1/16 inches long.

Fertility: Normal, viable seed is produced.

Behavior: Once the plant begins to bloom, it has a prolific blooming habit and yields a striking display of medium to large sized, single flowers of medium pink color that becomes lighter at the center and an extremely distinctive deep pink speckling pattern on each of the petals, against its medium sized, medium green leaves.

General observations: Inflorescence is of medium to large size and of somewhat open heads of double florets, which are in contrast to the medium green , zoned foliage of the plant, and measure up to about 3 1/4 inches in diameter. At the optimum stage of opening, florets are distinctive and striking with their medium pink coloration that becomes lighter at the center and a highly unique deep pink speckling pattern on each of the petals. Flowers have good heat tolerance when compared to other varieties.

### STEM

Thickness: Medium to thick.

Internodes: Average.

Branching: Very free breaking.

Attitude: Upright, bushy habit.

### FOLIAGE

Leaves

Quantity.—Abundant.

Shape.—Reniform.

Size.—Medium, largest leaves range from about 2 5/8 to 3 3/8 inches across.

Margin.—Crenate.

Texture.—Somewhat pubescent, matte texture.

Coloration.13 Dark green, RHS Green Group, 137 C, with a medium sized zone of RHS Green Group, 137 A. Zonation is centrally located on the leaf surface, and concentric to the point of the petiole attachment and with green surface portions both inside of the outside of the zonal band.

Petioles

Texture.—Lightly pubescent.

Lenqth.—Approximately 1 3/4 to 2 5/8 inches.

Color.—RHS Green Group, 143 C.

“Eleanor Byrd” is comparable to “Melody” with which it shares market class, but is easily distinguishable therefrom in terms of flower color; “Eleanor Byrd” having medium pink petal coloration that becomes lighter toward the center of each floret, as well as the very distinctive deep pink speckling pattern on each petal, while the florets of “Meldoy” are a deeper shade of pink with a small white eye. “Eleanor Byrd” is readily distinguished from “Fox” by having florets of much lighter petal coloration and the unique speckling effect, in addition to a slightly darker green leaf coloration and a more distinguishable leaf zonation.

I claim:

1. A new and distinct variety of Geranium plant, substantially as shown and described, characterized by its upright, bushy growth habit; its medium-sized, medium green foliage with light zonation; its medium pink single flowers that become lighter toward the center and have a very distinctive deep pink speckling pattern on each petal; its abundant blooming habit and its heat tolerance.
